(19) United States
(12) Patent Application Publication
(10) Pub. No.: US 2013/0143279 A1
(43) Pub. Date: Jun. 6, 2013

(54) PROCESS FOR PRODUCING METHIONINE

(71) Applicants: Koji Hagiya, Ibaraki-shi (JP); Hiroyuki Asako, Toyonaka-shi (JP)

(72) Inventors: Koji Hagiya, Ibaraki-shi (JP); Hiroyuki Asako, Toyonaka-shi (JP)

(73) Assignee: SUMITOMO CHEMICAL COMPANY, LIMITED, Chuo-ku, Tokyo (JP)

(21) Appl. No.: 13/700,891

(22) PCT Filed: May 30, 2011
(86) PCT No.: PCT/JP2011/062856

§ 371 Date: Jan. 7, 2013

(30) Foreign Application Priority Data
Jun. 1, 2010 (JP) 2010-125561
Feb. 17, 2011 (JP) 2011-031704

(51) Int. Cl. C07C 319/20; C07C 319/18

(52) U.S. Cl. CPC C07C 319/20; C07C 319/18
USPC 435/113; 562/526; 564/501

(57) ABSTRACT

The present invention relates to a process for producing methionine, comprising a first step of reacting 2-amino-3-buten-1-ol with methanethiol, and a second step of oxidizing 2-amino-4-methylthio-1-butanol obtained in the first step. The present invention also relates to a process for producing 2-amino-4-methylthio-1-butanol, comprising a step of reacting 2-amino-3-buten-1-ol with methanethiol.

### TECHNICAL FIELD

The present application is filed, claiming the priorities based on the Japanese Patent Application Nos. 2010-125561 (filed on Jun. 1, 2010) and 2011-031704 (filed on Feb. 17, 2011), and a whole of the contents of the applications is incorporated herein by reference.

The present invention relates to a process for producing methionine. The present invention also relates to a process for producing 2-amino-4-methylthio-1-butanol.

### BACKGROUND ART

Methionine (another name: 2-amino-4-(methylthio) butyric acid) is an essential amino acid, which is very useful for a feed additive.

As a process for producing methionine, a process in which 3-methylthiopropionaldehyde obtained by addition of methanethiol to acrolein is reacted with hydrogen cyanide and ammonium bicarbonate to obtain a substituted hydantoin; and then, the substituted hydantoin is hydrolyzed with an alkali, is known from, for example, “Industrial Organic Chemistry”, Tokyo Kagaku-Dojin, 1978, pp. 273-275.

### DISCLOSURE OF INVENTION

### Problem to be Solved by the Invention

In the above-described process, sodium cyanide is used as a raw material. Sodium cyanide is however needed to be handled under sufficient control in equipment adapted to such control.

Under such a circumstance, there has been demand for a new process by which methionine can be produced without using of sodium cyanide as a raw material.

### Means for Solving the Problem

As a result of the present inventors' intensive studies for solving the above-described problem, the present invention is accomplished.

The present invention provides the followings:

[1] A process for producing methionine, comprising a first step of reacting 2-amino-3-buten-1-ol with methanethiol, and a second step of oxidizing 2-amino-4-methylthio-1-butanol obtained in the first step.
[2] The process according to the above item [1], wherein the first step is a step of reacting 2-amino-3-buten-1-ol with methanethiol in the presence of a radical initiator.
[3] The process according to the above item [2], wherein the radical initiator is an azo compound.
[4] The process according to any one of the above items [1] to [3], wherein the first step is a step of reacting 2-amino-3-buten-1-ol with methanethiol in the presence of a solvent.
[5] The process according to the above item [4], wherein the solvent is an ester solvent.
[6] The process according to any one of the above items [1] to [5], wherein the second step is a step of oxidizing 2-amino-4-methylthio-1-butanol in the presence of at least one metal selected from the group consisting of copper and the elements belonging to Group 8, 9 or 10 of the periodic table.
[7] The process according to any one of the above items [1] to [5], wherein the second step is a step of oxidizing 2-amino-4-methylthio-1-butanol in the presence of copper and water.
[8] The process according to any one of the above items [1] to [5], wherein the second step is a step of oxidizing 2-amino-4-methylthio-1-butanol in the presence of oxygen and either ruthenium or platinum.
[9] The process according to any one of the above items [6] to [8], wherein the second step is a step of oxidizing 2-amino-4-methylthio-1-butanol further in the presence of at least one typical metal compound selected from the group consisting of alkali metal compounds and alkaline earth metal compounds.
[10] The process according to the above item [9], wherein the typical metal compound is an alkali metal hydroxide or an alkaline earth metal hydroxide.
[11] The process according to any one of the above items [1] to [5], wherein the second step is a step of oxidizing 2-amino-4-methylthio-1-butanol by an action of a microbial cell of a microorganism capable of converting 2-amino-4-methylthio-1-butanol into methionine or an action of a processed product of the microbial cell.
[12] The process according to the above item [11], wherein the microorganism is a microorganism cultured in a culture medium containing a lower aliphatic alcohol.
[13] The process according to the above item [12], wherein the lower aliphatic alcohol is a liner or branched aliphatic alcohol having 1 to 5 carbon atoms.
[14] The process according to any one of the above items [11] to [13], wherein the microorganism is at least one microorganism selected from the group consisting of the microorganisms belonging to the genus Alcaligenes, the microorganisms belonging to the genus Bacillus, the microorganisms belonging to the genus Pseudomonas, the microorganisms belonging to the genus Rhodobacter and the microorganisms belonging to the genus Rhodococcus.
[15] A process for producing 2-amino-4-methylthio-1-butanol, comprising a step of reacting 2-amino-3-buten-1-ol with methanethiol.
[16] The process according to the above item [15], wherein the above-described step is a step of reacting 2-amino-3-buten-1-ol with methanethiol in the presence of a radical initiator.
[17] The process according to the above item [16], wherein the radical initiator is an azo compound.
[18] The process according to any one of the above items [15] to [17], wherein the above-described step is a step of reacting 2-amino-3-buten-1-ol with methanethiol in the presence of a solvent.
[19] The process according to the above item [18], wherein the solvent is an ester solvent.

According to the present invention, methionine can be produced without using of sodium cyanide as a raw material.

### MODES FOR CARRYING OUT THE INVENTION

Hereinafter, the present invention will be described in detail.

The process for producing methionine according to the present invention comprises a first step of reacting 2-amino-3-buten-1-ol with methanethiol, and a second step of oxidizing 2-amino-4-methylthio-1-butanol obtained in the first step.

Furthermore, the process for producing 2-amino-4-methylthio-1-butanol according to the present invention comprises a step of reacting 2-amino-3-buten-1-ol with methanethiol (hereinafter sometimes referred to as “first step”).

Firstly, 2-amino-3-buten-1-ol for use in the first step is described.

### 2-amino-3-buten-1-ol

2-amino-3-buten-1-ol can be obtained, for example, by reacting 1,2-epoxy-3-butene with ammonia. Hereinafter, the reaction of 1,2-epoxy-3-butene with ammonia is sometimes referred to as “the present amination reaction”.

1,2-epoxy-3-butene for use in the present amination reaction can be produced by a known method in which an oxidant such as oxygen, an organic peroxide, or hydrogen peroxide is reacted with butadiene. Preferably, 1,2-epoxy-3-butene can be produced by a method of reacting oxygen with butadiene in the presence of a silver-containing catalyst. Such method can be found in JP 3-502330 A, for example.

The present amination reaction may be carried out by any of the following methods (A-1), (A-2) and (A-3).

### (A-1)

A method of reacting 1,2-epoxy-3-butene with ammonia water in the absence of a metal catalyst (e.g., Journal of the American Chemical Society, Vol. 79 pp 4792-4796, 1950);

### (A-2)

A method of reacting 1,2-epoxy-3-butene with ammonia in the presence of a Pd (zero-valence) complex and Lewis acid (e.g., U.S. Pat. No. 5,463,079).

### (A-3)

A method of reacting 1,2-epoxy-3-butene with ammonia in the presence of a compound which contains at least one element selected from the group consisting of lanthanoids and the elements belonging to Group 3 of the periodic table.

The present amination reaction is preferably carried out by the above-described method (A-3).

Hereinafter, the present amination reaction will be described based on an embodiment in which the method (A-3) is employed. However, the present amination reaction is not limited to this embodiment.

In the method (A-3), examples of the elements belonging to Group 3 of the periodic table include scandium, ytterium and lanthanum; and examples of the lanthanoids include cerium, samarium, europium, gadolinium and ytterbium.

The above-described element is preferably at least one element selected from the elements belonging to Group 3 of the periodic table, more preferably, at least one element selected from the group consisting of scandium, ytterium and lanthanum, still more preferably at least one element selected from the group consisting of scandium and ytterium.

Examples of the compound which contains at least one element selected from the group consisting of lanthanoids and the elements belonging to Group 3 of the periodic table include

scandium compounds such as scandium oxide, scandium triflate, scandium acetate, scandium chloride, scandium sulfate and scandium nitrate;
ytterium compounds such as ytterium oxide, ytterium triflate, ytterium acetate, ytterium chloride, ytterium sulfate and ytterium nitrate;
lanthanum compounds such as lanthanum oxide, lanthanum triflate, lanthanum acetate, lanthanum chloride, lanthanum sulfate and lanthanum nitrate;
cerium compounds such as cerium oxide, cerium triflate, cerium acetate, cerium chloride, cerium sulfate and cerium nitrate;
samarium compounds such as samarium oxide, samarium triflate, samarium acetate, samarium chloride, samarium sulfate and samarium nitrate;
europium compounds such as europium oxide, europium triflate, europium acetate, europium chloride, europium sulfate and europium nitrate;
gadolinium compounds such as gadolinium oxide, gadolinium triflate, gadolinium acetate, gadolinium chloride, gadolinium sulfate and gadolinium nitrate; and
ytterbium compounds such as ytterbium oxide, ytterbium triflate, ytterbium acetate, ytterbium chloride, ytterbium sulfate and ytterbium nitrate. Hereinafter, the compound which contains at least one element selected from the group consisting of lanthanoids and the elements belonging to Group 3 of the periodic table is sometimes referred to as “the present amination catalyst”.

The present amination catalyst is preferably a scandium compound, an ytterium compound or a lanthanum compound, more preferably a scandium compound or an ytterium compound, still more preferably a scandium compound, far still more preferably scandium triflate.

The present amination catalysts may be used alone or as a mixture of two or more kinds thereof.

The present amination catalyst may be a hydrate or an anhydride.

The present amination catalyst may be supported on a support (hereinafter sometimes referred to as a supported amination catalyst) or may not be supported thereon. The support includes at least one selected from the group consisting of activated carbon, alumina, silica, zeolite, diatomite and zirconium oxide. It is advantageous for such a support to have a larger surface area because the reactivity of the present amination reaction can be enhanced. The supported amination catalyst may be a commercially available product, or may be a catalyst obtained as follows: for example, a nitrate, sulfate, acetate, halide and/or oxide of at least one element selected from the group consisting of lanthanoids and the elements belonging to Group 3 of the periodic table is supported on the above-described support by coprecipitation method or impregnation method, and then this supported salt is calcined.

The amount of the present amination catalyst to be used is preferably 0.001 mol or more per mol of 1,2-epoxy-3-butene because a higher yield can be achieved. Although the upper limit is not limited, it is usually 0.5 mol or less per mol of 1,2-epoxy-3-butene.

The ammonia for use in the present amination reaction can be used in either form of liquid ammonia, an ammonia gas or an ammonia solution. Examples of the ammonia solution include ammonia water and an ammonia/methanol solution. The ammonia solution may be a commercially available product or may be a solution prepared by dissolving ammonia in a polar solvent such as water, or methanol.

As the ammonia, an ammonia solution is preferably used, and ammonia water is more preferably used.

The amount of the ammonia to be used is preferably one mol or more per mol of 1,2-epoxy-3-butene, and it is more preferably 5 mol or more, still more preferably 10 mol or more, per mol of 1,2-epoxy-3-butene, because a reaction of the resultant 2-amino-3-buten-1-ol with 1,2-epoxy-3-butene can be suppressed. Although an upper limit of this amount is not limited, it is usually 100 mol or less per mol of 1,2-epoxy-3-butene.

The present amination reaction may be carried out in the absence or presence of a solvent. Preferably, the present amination reaction is carried out in the presence of a solvent. Examples of the solvent include ether solvents such as diethyl ether, methyl-tert-butyl ether and tetrahydrofuran; halogen solvents such as chloroform and chlorobenzene; alcohol solvents such as methanol, ethanol, isopropanol and tert-butanol; nitrile solvents such as acetonitrile and propionitrile; and water. The solvent is preferably water. The amount of the solvent to be used is, while not limited to, preferably 100 parts by weight or less per part by weight of 1,2-epoxy-3-butene, because a volume efficiency can be improved.

The present amination reaction may be carried out under normal pressure or increased pressure. Preferably, the present amination reaction is carried out under the pressure of from about 0.3 to about 2 MPa.

The reaction temperature is preferably from −20 to 150° C., more preferably from 0 to 100° C. When the reaction temperature is not higher than 150° C., the generation of byproducts can be suppressed. When the reaction temperature is not lower than −20° C., the reactivity of the present amination reaction can be enhanced.

The present amination reaction is carried out, for example, by mixing 1,2-epoxy-3-butene, ammonia and the present amination catalyst in the presence or absence of a solvent. While the order of mixing the reaction reagents in the present amination reaction is not limited, such mixing is preferably carried out by the following method (A-3-1) or (A-3-2).

### (A-3-1)

A method comprising the steps of mixing ammonia with the present amination catalyst in the presence or absence of a solvent, and adding 1,2-epoxy-3-butene to the resulting mixture.

### (A-3-2)

A method comprising the steps of mixing 1,2-epoxy-3-butene with ammonia in the presence or absence of a solvent, and adding the present amination catalyst to the resulting mixture.

When the present amination reaction is carried out by the method (A-3-1) under normal pressure, 1,2-epoxy-3-butene is preferably added dropwise to the resulting mixture. When the present amination reaction is carried out by the method (A-3-1) under increased pressure, 1,2-epoxy-3-butene is added preferably by injection.

The degree of the reaction progress can be confirmed by analyzing means such as gas chromatography, high-performance liquid chromatography, thin-layer chromatography, nuclear magnetic resonance spectrum analysis, or infrared-absorption spectrum analysis.

After completion of the reaction, 2-amino-3-buten-1-ol may be brought out by a procedure in which ammonia is optionally recovered from the reaction mixture, and then, the present amination catalyst is removed by filtration, after that, the filtrate is concentrated, separated and crystallized.

In another embodiment, 2-amino-3-buten-1-ol may be brought out by a procedure in which ammonia is optionally recovered from the reaction mixture, and then, the present amination catalyst is separated by filtration, after that, the filtrate is mixed with an acid such as oxalic acid to form a salt, and the resultant salt is crystallized. Such method can be found in, for example, Journal of the American Chemical Society, Vol. 79, pp 4792-4796, 1950.

In a different embodiment, 2-amino-3-buten-1-ol may be brought out by a procedure in which ammonia is optionally recovered from the reaction mixture, and then, the present amination catalyst is separated by filtration, and the filtrate optionally concentrated is rectified.

The present amination catalyst separated by filtration from the reaction mixture can be recycled for the present amination reaction as it is. Alternatively, the present amination catalyst separated by filtration from the reaction mixture can be recycled for the present amination reaction after it is purified as necessary. When the present amination catalyst is contained in a solution obtained by the liquid-separation treatment, the catalyst recovered by concentrating and purifying the solution may be recycled for the present amination.

The obtained 2-Amino-3-buten-1-ol may be directly subjected to the first step or may be subjected to the first step after distilled or purified by column chromatography or other purifying means. The reaction mixture may be directly subjected to the first step without bringing out 2-amino-3-buten-1-ol therefrom.

Next, the first step will be described.

### <First Step>

2-Amino-3-buten-1-ol is reacted with methanethiol. Hereinafter, this reaction is sometimes referred to as “the present addition reaction”. By the present addition reaction, 2-amino-4-methylthio-1-butanol is obtained.

Methanethiol for use in the present addition reaction may be a commercially available product or may be prepared by a known method, for example, a reaction of methanol with hydrogen sulfide.

The amount of methanethiol to be used is preferably one mol or more per mol of 2-amino-3-buten-1-ol. An upper limit of this amount is, while not limited to, usually 20 mol or less per mol of 2-amino-3-buten-1-ol. The amount of methanethiol to be used at the start of the present addition reaction is preferably 4 mol or less per mol of 2-amino-3-buten-1-ol, because the start of the present addition reaction can easily be controlled.

The present addition reaction is preferably carried out in the presence of a radical initiator so as to obtain 2-amino-4-methylthio-1-butanol in a high yield.

Hereinafter, the present addition reaction will be described based on an embodiment in which the reaction is carried out in the presence of a radical initiator. However, the present addition reaction is not limited to this embodiment.

Examples of the radical initiator include halogen molecules, organic peroxides, azo compounds, triethylborane and diethylzinc.

Examples of the halogen molecule include chlorine. Examples of the organic peroxide include di-tert-butyl peroxide, tert-butylhydro peroxide and benzoyl peroxide. Examples of the azo compound include azo nitrile compounds such as 2,2′-azobisisobutylonitrile, 2,2′-azobis(2,4-dimethylvaleronitrile), 2,2′-azobis(2-methylbutylonitrile), 1,1′-azobis(cyclohexane-1-carbonitrile), 2,2′-azobis(4-methoxy-2,4-dimethylvaleronitrile), 4,4′-azobis-4-cyanopentanoic acid, 2-phenylazo-2,4-dimethyl-4-methoxyvaleronitrile and 2-cyano-2-propylazoformamide; azo ester compounds such as azobisisobutanol diacetate, methyl azobisisobutyrate and ethyl azobisisobutyrate; azoamidine compounds such as 2,2′-azobis(2-amidinopropane)-dihydrochloride; azoimidazoline compounds such as 2,2′-azobis[2-(2-imidazoline-2-yl)propane]; azoamide compounds such as 1,1′-azobisformamide, 1,1′-azobis(N-methylformamide) and 1,1′-azobis(N,N-dimethylformamide); and azoalkyl compounds such as azo-tert-butane.

The radical initiator is preferably an azo compound, more preferably an azonitrile compound, an azo ester compound, an azoamidine compound or an azoimidazoline compound, still more preferably an azonitrile compound, because of ease of availability.

The amount of the radical initiator to be used is preferably 0.001 mol or more per mole of 2-amino-3-buten-1-ol. The upper limit of this amount is, while not limited to, usually 0.2 mol or less per mole of 2-amino-3-buten-1-ol.

The present addition reaction may be carried out in the absence or presence of a solvent. Preferably, the present addition reaction is carried out in the presence of a solvent. The solvent to be used is such one that does not inhibit the present addition reaction. Examples of the solvent include hydrocarbon solvents such as hexane, heptane and toluene; halogenated hydrocarbon solvents such as chlorobenzene and chloroform; ester solvents such as ethyl acetate; tertiary alcohol solvents such as tert-butylalcohol; nitrile solvents such as acetonitrile and propionitrile; and water. The solvent is preferably an ester solvent. These solvents may be used alone or as a mixture of two or more kinds thereof.

The amount of the solvent to be used is, while not limited to, preferably 100 parts by weight or less per part of 2-amino-3-buten-1-ol, because the volume efficiency can be improved.

The reaction temperature may vary depending on the kind or amount of the radical initiator to be used, and is preferably from −10 to 100° C., more preferably from 0 to 50° C. When the reaction temperature is not lower than −10° C., the present addition reaction can be carried out at a higher rate. When the reaction temperature is not higher than 100° C., the generation of byproducts can be suppressed.

The present addition reaction may be carried out under reduced pressure, normal pressure or increased pressure. Preferably, the present addition reaction is carried out under normal pressure or increased pressure, since methanethiol, the boiling point of which is 6° C., tends to be volatile under a reduced pressure.

The present addition reaction may be carried out by mixing 2-amino-3-buten-1-ol with methanethiol in the presence of a radical initiator. The mixing method is not limited.

When the present addition reaction is carried out under normal pressure, for example, the following method (1-1) may be employed.

(1-1)

A method comprising the steps of mixing 2-amino-3-buten-1-ol with a radical initiator, controlling the temperature of the resulting mixture to be at the reaction temperature, and blowing a gaseous methanethiol into the mixture.

When the present addition reaction is carried out under increased pressure, for example, the following method (1-2) or (1-3) may be employed.

(1-2)

A method comprising the steps of charging a sealable vessel such as an autoclave with a radical initiator and 2-amino-3-buten-1-ol, controlling the temperature of the mixture to be at the reaction temperature after closing the vessel, and injecting a gaseous methanethiol into the mixture.

(1-3)

A method comprising the steps of mixing a radical initiator, 2-amino-3-buten-1-ol and methanethiol in a sealable vessel such as an autoclave at not higher than the boiling point of methanethiol, and controlling the temperature of the mixture to be at the reaction temperature after closing the vessel.

The degree of the reaction progress can be confirmed by analyzing means such as gas chromatography, high-performance liquid chromatography, thin-layer chromatography, nuclear magnetic resonance spectrum analysis, or infrared-absorption spectrum analysis.

After completion of the reaction, 2-amino-4-methylthio-1-butanol may be brought out by a procedure in which methanethiol and/or the radical initiator and a decomposition product thereof are optionally removed from the resultant reaction mixture, and the residue is concentrated, and then 2-amino-3-buten-1-ol is optionally removed. 2-Amino-4-methylthio-1-butanol may be brought out by precipitating as an acid addition salt with an acid such as hydrochloric acid, or sulfuric acid, and treating the resultant acid addition salt with a base such as sodium hydroxide, or ammonia.

As the method of removing 2-amino-3-buten-1-ol, for example, a distillation treatment can be employed. After the 2-Amino-3-buten-1-ol removed by distillation is recovered and optionally purified, the recovered 2-Amino-3-buten-1-ol may be recycled for the present addition reaction.

As the method of removing methanethiol, for example, a method in which methanethiol is distilled off from the reaction mixture under reduced pressure, or a method in which an inert gas is blown into the reaction mixture to evaporate methanethiol, can be employed. After the removed methanethiol is recovered and optionally purified, the recovered methanethiol may be recycled for the present addition reaction.

As the method of removing the radical initiator and the decomposed product thereof, depending on the kind of the radical initiator used in the present addition reaction, for example, any of the following methods can be employed: A method in which the reaction mixture is mixed with a polar solvent to precipitate the radical initiator and its decomposition product, and the precipitate is filtered; A method in which the reaction mixture is mixed with a polar solvent and a non-polar solvent, and the radical initiator and its decomposition product distributed in a non-polar solvent phase are removed; A method in which a polar solvent incompatible with water, water and the reaction mixture are mixed, and the radical initiator and its decomposition product distributed in a water phase are removed therefrom.

Examples of the polar solvent for use in such methods include water and a solvent mixture of water and an alcohol (e.g., methanol, or ethanol). Examples of the non-polar solvent include hydrocarbon solvents such as hexane, heptane, toluene and xylene. Examples of the polar solvent incompatible with water include ester solvents such as ethyl acetate, and ether solvents such as methyl tert-butyl ether and diisopropyl ether. Amounts of the polar solvent, the non-polar solvent and the polar solvent incompatible with water to be used are not limited. When the present addition reaction is carried out in the presence of these solvents, any of these solvents may be additionally added during the reaction. After the removed radical initiator is recovered and optionally purified, the recovered radical initiator may be recycled for the present addition reaction.

The obtained 2-amino-4-methylthio-1-butanol may be directly subjected to the second step, or may be purified by distillation, column chromatography or other purifying means and then may be subjected to the second step. The reaction mixture may be directly subjected to the second step without bringing out 2-amino-4-methylthio-1-butanol.

Next, the second step will be described.

### <Second Step>

The 2-amino-4-methylthio-1-butanol obtained in the first step is oxidized. Hereinafter, the oxidation of the 2-amino-4-methylthio-1-butanol is sometimes referred to as “the present oxidation reaction”. By the present oxidation reaction, methionine is obtained. The present oxidation reaction may be carried out in the presence of a metal catalyst. Alternatively, the present oxidation reaction may be carried out by an action of a microbial cell of a microorganism capable of converting 2-amino-4-methylthio-1-butanol into methionine or by an action of a processed product of the microorganism. Hereinafter, the present oxidation reaction in the former case is sometimes referred to as “the present oxidation reaction 1”, and the present oxidation reaction in the latter case is sometimes referred to as “the present oxidation reaction 2”.

Preferably, the present oxidation reaction 1 is carried out by oxidizing 2-amino-4-methylthio-1-butanol in the presence of at least one metal selected from the group consisting of copper and the elements belonging to Group 8, 9 or 10 of the periodic table. More preferably, the present oxidation reaction 1 is carried out by the following method (2-1) or (2-2).

(2-1)

A method for oxidizing 2-amino-4-methylthio-1-butanol, wherein the oxidation is carried out in the presence of oxygen and at least one metal selected from the group consisting of the elements belonging to Group 8, 9 or 10 of the periodic table.

(2-2)

A method for oxidizing 2-amino-4-methylthio-1-butanol, wherein the oxidation is carried out in the presence of copper and water.

Hereinafter, the present oxidation reaction 1 will be described based on the embodiments by the methods (2-1) and (2-2). However, the present oxidation reaction 1 is not limited to these embodiments.

The embodiment by the method (2-1) will be described.

Examples of the elements of Group 8 of the periodic table include iron, ruthenium and the like. Examples of the elements of Group 9 of the periodic table include cobalt, rhodium and the like. Examples of the elements of Group 10 of the periodic table include nickel, palladium, platinum and the like. At least one metal selected from the group consisting of the elements belonging to Group 8, 9 or 10 of the periodic table is preferably ruthenium or platinum, more preferably platinum. Hereinafter, at least one metal selected from the group consisting of the elements belonging to Group 8, 9 or 10 of the periodic table is sometimes referred to as the oxygen-oxidation catalyst.

The oxygen-oxidation catalyst may be supported on a support (hereinafter, such a catalyst is sometimes referred to as a supported oxygen-oxidation catalyst), or may not be supported thereon. Alternatively, the oxygen-oxidation catalyst may be a catalyst in which an alloy containing at least one metal selected from the group consisting of the elements belonging to Group 8, 9 or 10 of the periodic table is treated with an acid or an alkali (hereinafter, such a catalyst sometimes referred to as a developing oxygen-oxidation catalyst).

The support includes at least one selected from the group consisting of activated carbon, alumina, silica, zeolite, diatomite and zirconium oxide. It is advantageous for such a support to have a larger surface area because the reactivity of the reaction can be enhanced. The supported oxygen-oxidation catalyst may be a commercially available product, or may be a catalyst obtained as follows: for example, at least one compound selected from the group consisting of nitrates, sulfates, formates, acetates, carbonates, halides, hydroxides and oxides of at least one element selected from the group consisting of the elements belonging to Group 8, 9 or 10 of the periodic table is supported on the above-described support by coprecipitation method or impregnation method, and then this supported compound is calcined or reduced with hydrogen.

The oxygen-oxidation catalyst is preferably a developing oxygen-oxidation catalyst or a supported oxygen-oxidation catalyst, more preferably a supported oxygen-oxidation catalyst.

The amount of the oxygen-oxidation catalyst to be used may vary depending on the form of the oxygen-oxidation catalyst in use, and is preferably 0.001 mol or more, more preferably from 0.001 to 0.5 mol per mole of 2-amino-4-methylthio-1-butanol from an economical viewpoint.

The oxygen may be an oxygen gas, or an oxygen gas diluted with an inert gas such as nitrogen, or oxygen in an air. Besides, oxygen in an air may be diluted with an inert gas such as nitrogen for use as the above-described oxygen.

The amount of the oxygen to be used is preferably one mole or more per mole of 2-amino-4-methylthio-1-butanol, and the upper limit of this amount is not limited.

Preferably, the present oxidation reaction 1 is carried out further in the presence of at least one typical metal compound selected from the group consisting of alkali metal compounds and alkaline earth metal compounds.

Examples of the alkali metal compounds include alkali metal carbonates such as sodium carbonate, sodium bicarbonate, potassium carbonate, potassium bicarbonate, lithium carbonate and lithium bicarbonate; and alkali metal hydroxides such as sodium hydroxide, potassium hydroxide and lithium hydroxide.

Examples of the alkaline earth metal compounds include alkaline earth metal carbonates such as magnesium carbonate and calcium carbonate; and alkaline earth metal hydroxides such as magnesium hydroxide and calcium hydroxide.

The typical metal compound is preferably an alkali metal hydroxide and an alkaline earth metal hydroxide, more preferably an alkali metal hydroxide, still more preferably sodium hydroxide.

The amount of the typical metal compound to be used is preferably one mol or more per mol of 2-amino-4-methylthio-1-butanol, while an upper limit thereof is not limited. The amount of the typical metal compound to be used is usually 2 mol or less per mol of 2-amino-4-methylthio-1-butanol.

Preferably, the present oxidation reaction 1 is carried out further in the presence of a solvent.

There is no limit in selection of the solvent insofar as it does not inhibit the present oxidation reaction 1. Examples of such a solvent include ester solvents such as ethyl acetate, nitrile solvents such as acetonitrile and propionitrile, water, and mixtures thereof. The solvent is preferably water, a mixture of water and an ester solvent or a mixture of water and a nitrile solvent, more preferably a mixture of water and a nitrile solvent, still more preferably a mixture of water and acetonitrile.

The amount of the solvent to be used is usually 100 parts by weight or less per part by weight of 2-amino-4-methylthio-1-butanol, while this amount is not limited.

In the present oxidation reaction 1, the order of mixing the reaction reagents is not limited. In the preferable embodiment, for example, 2-amino-4-methylthio-1-butanol, an oxygen-oxidation catalyst, a typical metal compound and a solvent are mixed, and then the resulting mixture is mixed with oxygen.

The present oxidation reaction 1 may be carried out under reduced pressure, normal pressure or increased pressure. Preferably, this reaction is carried out under normal pressure or increased pressure.

A temperature for the present oxidation reaction 1 may vary depending on an amount of the oxygen-oxidation catalyst to be used, an amount of the oxygen to be used or the like, and is preferably from 0 to 150° C., more preferably from 20 to 100° C. When the reaction temperature is not lower than 0° C., the oxidation reaction can be carried out at higher rate. When the reaction temperature is not higher than 150° C., the oxidation reaction can be carried out in higher selectivity.

Proceeding of the present oxidation reaction 1 can be confirmed by analyzing means such as gas chromatography, high-performance liquid chromatography, thin-layer chromatography, nucleic magnetic resonance spectrum analysis, or infrared absorption spectrum analysis.

After completion of the present oxidation reaction 1, for example, the methionine may be brought out by a procedure in which the resultant reaction mixture is filtered to remove the oxygen-oxidation catalyst, and then, the filtrate is optionally neutralized with a mineral acid such as sulfuric acid or hydrochloric acid and is then concentrated and cooled.

The methionine thus brought out may be purified by distillation, column chromatography, crystallization or other purifying means.

The embodiment by the method (2-2) will be described.

Copper (hereinafter sometimes referred to as a copper catalyst) may be supported on a carrier (hereinafter this catalyst is sometimes referred to as a supported copper catalyst) or may not be supported thereon. Alternatively, the copper obtained by treating a copper-containing alloy with an acid or an alkali (hereinafter this catalyst is sometimes referred to as a developing copper catalyst) may be used.

The support includes at least one support selected from the group consisting of activated carbon, alumina, silica, zeolite, diatomite and zirconium oxide. It is advantageous for such a support to have a larger surface area because the reactivity of the reaction can be enhanced. The supported copper catalyst may be a commercially available product, or may be a catalyst in which copper or an alloy of copper and aluminum is supported on the above-described support, or may be a catalyst obtained as follows: for example, at least one copper compound selected from the group consisting of copper nitrates, copper sulfates, copper formates, copper acetates, copper carbonates, copper halides, copper hydroxides and copper oxides is supported on the above-described support by coprecipitation method or impregnation method, and then this supported compound is calcined or reduced with hydrogen. The developing copper catalyst, in other words “sponge catalyst” may be a commercially available product, or may be a catalyst obtained by techniques known to those skilled in the art from various alloys. The developing copper catalyst includes a catalyst prepared from alloys containing copper and aluminum, such as Raney copper catalyst described in U.S. Pat. No. 5,292,936.

The copper catalyst is preferably a developing copper catalyst or a supported copper catalyst, more preferably a developing copper catalyst.

The amount of the copper catalyst to be used may vary depending on the form of the copper catalyst in use, and is preferably 0.001 mol or more per mol of 2-amino-4-methylthio-1-butanol. Economically preferred amount is 0.5 mol or less per mol of 2-amino-4-methylthio-1-butanol.

The amount of water to be used is preferably one mol or more per mol of 2-amino-4-methylthio-1-butanol. While an upper limit thereof is not limited, preferably it is 100 mol or less per mol of 2-amino-4-methylthio-1-butanol.

Preferably, the present oxidation reaction 1 is carried out further in the presence of at least one typical metal compound selected from the group consisting of alkali metal compounds and alkaline earth metal compounds.

Examples of the alkali metal compounds include alkali metal carbonates such as sodium carbonate, sodium bicarbonate, potassium carbonate, potassium bicarbonate, lithium carbonate and lithium bicarbonate; and alkali metal hydroxides such as sodium hydroxide, potassium hydroxide and lithium hydroxide.

Examples of the alkaline earth metal compounds include alkaline earth metal carbonates such as magnesium carbonate and calcium carbonate; and alkaline earth metal hydroxides such as magnesium hydroxide and calcium hydroxide.

The typical metal compound is preferably an alkali metal hydroxide or an alkaline earth metal hydroxide, more preferably an alkali metal hydroxide, still more preferably sodium hydroxide.

The amount of the typical metal compound to be used is preferably one mol or more per mol of 2-amino-4-methylthio-1-butanol, while an upper limit thereof is not limited, usually 2 mol or less per mol of 2-amino-4-methylthio-1-butanol.

The present oxidation reaction 1 may be carried out further in the presence of an organic solvent.

There is no limit in selection of the organic solvent insofar as it does not inhibit the present oxidation reaction 1. Examples of such a solvent include ester solvents such as ethyl acetate, and nitrile solvents such as acetonitrile and propionitrile.

The amount of the organic solvent to be used is usually 100 parts by weight or less per part by weight of 2-amino-4-methylthio-1-butanol, while this amount is not limited.

In the present oxidation reaction 1, the order of mixing the reaction reagents is not limited. In the preferable embodiment, for example, 2-amino-4-methylthio-1-butanol, a typical metal compound and water are mixed, and then the resulting mixture is admixed with a copper catalyst. This mixing is preferably carried out under an atmosphere of an inert gas such as nitrogen.

The present oxidation reaction 1 may be carried out under reduced pressure, normal pressure and increased pressure. Preferably, this reaction is carried out under normal pressure or increased pressure.

A temperature for the present oxidation reaction 1 may vary depending on a kind and an amount of the copper catalyst to be used, and is preferably from 0 to 200° C., more preferably from 50 to 180° C. When the reaction temperature is not lower than 0° C., the oxidation reaction rate can be higher. When the reaction temperature is not higher than 200° C., the oxidation reaction can be carried out in higher selectivity.

Proceeding of the present oxidation reaction 1 can be confirmed by analyzing means such as gas chromatography, high-performance liquid chromatography, thin-layer chromatography, nucleic magnetic resonance spectrum analysis, or infrared absorption spectrum analysis.

After completion of the present oxidation reaction 1, for example, the methionine may be brought out by a procedure in which the resultant reaction mixture is filtered to remove copper catalyst, and then, the filtrate is optionally neutralized with a mineral acid such as sulfuric acid or hydrochloric acid and is then concentrated and cooled.

The obtained methionine may be purified by distillation, column chromatography, crystallization or other purifying means.

The present oxidation reaction 2 is carried out by an action of a microbial cell of a microorganism capable of converting 2-amino-4-methylthio-1-butanol into methionine or by an action of a processed product of the microbial cell. The microorganism is preferably a microorganism cultured in a culture medium containing a lower aliphatic alcohol.

Examples of the “lower aliphatic alcohol” to be used for the culture medium include a liner or branched aliphatic alcohols having 1 to 5 carbon atoms. Specific examples thereof include methanol, ethanol, 1-propanol, 2-propanol, 1-butanol, tert-butanol, 2-methyl-1-propanol, 2,2-dimethyl-1-propanol, 1,2-butanediol and 1,3-butanediol. Among them, 1-propanol, 1-butanol, 2,2-dimethyl-1-propanol, 1,2-butanediol and 1,3-butanediol are preferably used. Any of these lower aliphatic alcohols may be mixed in the culture medium at an appropriate ratio.

A method for culturing the microorganism in a culture medium containing a lower aliphatic alcohol will be described later.

The microorganism for the present oxidation reaction 2 preferably a microorganism capable of preferentially oxidizing a hydroxyl group of 2-amino-4-methylthio-1-butanol. The term “preferentially oxidizing” herein used means that oxidation of a hydroxyl group of a sulfur-containing amino alcohol compound proceed preferentially to oxidation of a sulfide of the same compound. Examples of the microorganism having such an ability (hereinafter sometimes referred to as “the present microorganism”) include at least one microorganism selected from the group consisting of the microorganisms belonging to Alcaligenes, the microorganisms belonging to the genus Bacillus, the microorganisms belonging to the genus Pseudomonas, the microorganisms belonging to the genus Rhodobacter and the microorganisms belonging to the genus Rhodococcus.

Specific examples of the microorganism for the present oxidation reaction 2 include at least one microorganism selected from the group consisting of the following microorganisms.

### <Group of Microorganisms>

Alcaligenes denitrificans, Alcaligenes eutrophus, Alcaligenes faecalis, Alcaligenes sp., Alcaligenes xylosoxydans, Bacillus alvey, Bacillus badius, Bacillus brevis, Bacillus cereus, Bacillus coagulans, Bacillus firmus, Bacillus licheniformis, Bacillus moritai, Bacillus pumilus, Bacillus sphaericus, Bacillus subtilis, Bacillus validus, Pseudomonas denitrificans, Pseudomonas ficuserectae, Pseudomonas fragi, Pseudomonas mendocina, Pseudomonas oleovorans, Pseudomonas ovalis, Pseudomonas pseudoalcaligenes, Pseudomonas putida, Pseudomonas putrefaciens, Pseudomonas riboflavina, Pseudomonas straminea, Pseudomonas syringae, Pseudomonas tabaci, Pseudomonas taetrolens, Pseudomonas vesicularis, Rhodobacter sphaeroides, Rhodococcus erythropolis, Rhodococcus groberulus, Rhodococcus rhodochrous and Rhodococcus sp.

Further, preferable as the present microorganism is, for example, at least one microorganism selected from the group consisting of the following microorganisms.

### <Group of Preferable Microorganisms>

Alcaligenes denitrificans JCM5490, Alcaligenes eutrophus ATCC43123, Alcaligenes faecalis IFO12669, Alcaligenes sp. IFO14130, Alcaligenes xylosoxydans IFO15125t, Alcaligenes xylosoxydans IFO15126t, Bacillus alvey IFO3343t, Bacillus badius ATCC14574t, Bacillus brevis JCM2503t, Bacillus cereus JCM2152t, Bacillus coagulans JCM2257t, Bacillus firmus JCM2512t, Bacillus licheniformis ATCC27811, Bacillus licheniformis IFO12197, Bacillus licheniformis IFO12200t, Bacillus moritai ATCC21282, Bacillus pumilus IFO12092t, Bacillus sphaericus IFO3341, Bacillus sphaericus IFO3526, Bacillus subtilis ATCC14593, Bacillus subtilis ATCC15841, Bacillus subtilis IFO3108, Bacillus subtilis IFO3132, Bacillus subtilis IFO3026, Bacillus subtilis IFO3037, Bacillus subtilis IFO3108, Bacillus subtilis IFO3134, Bacillus validus IFO13635, Pseudomonas denitrificans IAM1426, Pseudomonas denitrificans IAM1923, Pseudomonas ficuserectae JCM2400t, Pseudomonas fragi IAM12402, Pseudomonas fragi IFO3458t, Pseudomonas mendocina IFO14162, Pseudomonas oleovorans IFO13583t, Pseudomonas ovalis IFO12688, Pseudomonas pseudoalcaligenes JCM5968t, Pseudomonas putida IFO12996, Pseudomonas putida IFO14164t, Pseudomonas putida IFO3738, Pseudomonas putida IFO12653, Pseudomonas putrefaciens IFO3910, Pseudomonas riboflavina IFO13584t, Pseudomonas straminea JCM2783t, Pseudomonas syringae IFO14055, Pseudomonas tabaci IFO3508, Pseudomonas taetrolens IFO3460, Pseudomonas vesicularis JCM1477t, Rhodobacter sphaeroides ATCC17023, Rhodococcus erythropolis IFO12320, Rhodococcus groberulus ATCC15076, Rhodococcus rhodochrous ATCC15076, Rhodococcus rhodochrous ATCC15610, Rhodococcus rhodochrous ATCC19067, Rhodococcus rhodochrous ATCC19149, Rhodococcus rhodochrous ATCC19150, Rhodococcus rhodochrous ATCC21197, Rhodococcus rhodochrous ATCC21199, Rhodococcus rhodochrous JCM3202t, Rhodococcus sp. ATCC19070, Rhodococcus sp. ATCC19071, and Rhodococcus sp. ATCC19148.

The strains of these microorganisms may be separated from natural ones, or are easily available from the culture collections.

As such culture collections which these strains can be purchased, for example, the following are exemplified.

### 1. Institute of Fermentation Osaka (or IFO)

Presently, the strains are handled by the Biological Resource Center (or NBRC) of the National Institute of Technology and Evaluation, an independent administrative agency, and they are available at NBRC website (URL: http://www.nbrc.nite.go.jp/NBRC2/NBRCDispSearchServlet?lang=jp).

### 2. American Type Culture Collection (or ATCC)

The stains are handled by the ATCC business group of Summit Pharmaceuticals International Corporation, and they are available at ATCC website (URL: http://www.summitpharma.co.jp/japanese/service/s_ATCC.html).

### 3. Japan Collection of Microorganisms (or JCM)

Presently, control of the strains is transferred to the microbial material development section of the Bio Resource Center of RIKEN (or RIKEN BRC), an independent administrative agent, and the strains are available at JCM website (URL: http://www.jcm.riken.go.jp/JCM/aboutJCM_J.shtml).

### 4. IAM Culture Collection

Presently, strains of bacteria, yeasts and filamentous bacteria out of the strains of the IAM culture collection are transferred to the microbial material development section of the Bio Resource Center of RIKEN, an independent administrative agent; and strains of micro alga are transferred to the microorganism collection of the National Institute for Environmental Studies (or NIES), an independent administrative agent. The strains are available at the JCM or NIES website (URL: http://www.jcm.riken.go.jp/JCM/aboutJCM_J.shtml, http://mcc.nies.go.jp/aboutOnlineOrder.do)

The microbial cells of the microorganism capable of preferentially oxidize a hydroxyl group of the 2-amino-4-methylthio-1-butanol and the processed product thereof are available or can be prepared by screening a microorganism capable of converting 2-amino-4-methylthio-1-butanol into methionine. Specifically, for example, a test tube is charged with a sterilized culture medium (5 ml), and the cells available from the culture collection or cells purely separated from soil are inoculated thereon. The cells in the test tube are subjected to shaking culture at 30° C. under an aerobic condition. After completion of the culture, the cells are recovered by centrifugal separation to obtain viable cells. After a screw-top test tube is charged with 0.1M Tris-glycine buffer (pH 10) (2 ml), the viable cells are added, and they are suspended in each other. Methioninol (2 mg) is added to the suspension, and the resulting mixture is shaken at 30° C. for 3 to 7 days.

After completion of the reaction, 1 ml of the reaction liquid is sampled. The cells are removed from this sampling liquid, and then, an amount of produced methionine is analyzed by liquid chromatography.

In this way, a microorganism which has an ability to preferentially oxidize a hydroxyl group of the 2-amino-4-methylthio-1-butanol can be screened.

Furthermore, the microbial cells of the microorganism capable of preferentially oxidize a hydroxyl group of the 2-amino-4-methylthio-1-butanol and the processed product thereof are available or can be prepared by screening a microorganism cultured in a culture medium containing a lower aliphatic alcohol and capable of converting 2-amino-4-methylthio-1-butanol into methionine. Such screening may be conducted as follows: A test tube is charged with a sterilized culture medium (5 ml) which contains a lower aliphatic alcohol and which has been prepared by adding, to water (1 L), a lower aliphatic alcohol (5 g), polypeptone (5 g), yeast extract (3 g), meat extract (3 g), ammonium sulfate (0.2 g), potassium dihydrogenphosphate (1 g) and magnesium sulfate heptahydrate (0.5 g), and adjusting the pH of the mixture to 7.0. Then, cells available from the culture collections or cells purely separated from soil are inoculated on this culture medium. The cells in the test tube are subjected to shaking culture at 30° C. under an aerobic condition. After completion of the culture, the cells are recovered by centrifugal separation to obtain viable cells. After a screw-top test tube is charged with 0.1M Tris-glycine buffer (pH 10) (2 ml), the viable cells are added, and they are suspended in each other. Methioninol (2 mg) is added to the suspension, and the resulting mixture is shaken at 30° C. for 3 to 7 days.

After completion of the reaction, 1 ml of the reaction liquid is sampled. The cells are removed from this sampling liquid, and then, an amount of produced methionine is analyzed by liquid chromatography.

On the other hand, methionine is produced in the same manner as mentioned above, except that microbial cells have been cultured in a culture medium not containing lower aliphatic alcohol. Then the amount of the produced methionine is analyzed, and the resultant analyzed value is compared with the former amount of the produced methionine produced by the microbial cells cultured in a culture medium containing lower aliphatic alcohol, to thereby select a microorganism showing an activity to preferentially oxidize a hydroxyl group.

Next, a method for growing the present microorganism will be described.

The present microorganism may be cultured in a culture medium for use in growth of a variety of microorganisms, which contains a carbon source, a nitrogen source, an organic salt, an inorganic salt and the like.

Examples of the carbon source include saccharides such as glucose, dextrin and sucrose; sugar alcohols such as glycerol; organic acids such as fumaric acid, citric acid and pyruvic acid; animal oils; vegetable oils; and molasses. The amount of these carbon sources to be added to the culture medium is usually from about 0.1 to about 30% (w/v) of the culture solution.

Examples of the nitrogen source include natural organic nitrogen sources such as meat extract, peptone, yeast extract, malt extract, soybean flour, corn steep liquor, cottonseed flour, dry yeast and casamino acids; amino acids; sodium salts with inorganic acids such as sodium nitrate; ammonium salts with inorganic acids such as ammonium chloride, ammonium sulfate and ammonium phosphate; ammonium salts with organic acids such as ammonium fumarate and ammonium citrate; and urea. Among these nitrogen sources, the ammonium salts with organic acids, the natural organic nitrogen sources and amino acids may be used also as carbon sources in many cases. The amount of these nitrogen sources to be added to the culture medium is usually from about 0.1 to about 30% (w/v) of the culture solution.

Examples of the organic salt and the inorganic salt include chlorides, sulfates, acetates, carbonates and phosphates of potassium, sodium, magnesium, iron, manganese, cobalt, zinc or the like. Specific examples thereof are sodium chloride, potassium chloride, magnesium sulfate, ferrous sulfate, manganese sulfate, cobalt chloride, zinc sulfate, copper sulfate, sodium acetate, calcium carbonate, potassium hydrogenphosphate and potassium dihydrogenphosphate. The amount of these organic salts and/or inorganic salts to be added to the culture medium is usually from about 0.0001 to about 5% (w/v) of the culture solution.

As the culture method, solid culture and liquid culture (e.g., test-tube culture, flask culture, and jar fermenter culture) are exemplified.

There is no particular limit in selection of a culture temperature and pH of a culture solution, insofar as these conditions enable growth of the present microorganism. For example, a culture temperature is from about 15° C. to about 45° C. and a pH of a culture solution is from about 4 to about 8. While a culture time may be optionally selected depending on culture conditions, it is usually from about 1 day to about 7 days.

Next, a method for culturing the microbial cells of the present microorganism in a culture medium containing a lower aliphatic alcohol will be described.

The present microorganism may be cultured in a culture medium for culturing a variety of microorganisms, which culture medium appropriately contains a carbon source, nitrogen source, organic salt, inorganic salt or the like. As the carbon source for use in the culture medium, a lower aliphatic alcohol alone may be used, or a mixture system of carbohydrate, hydrocarbon, organic acid, sugar alcohol or the like may be used.

As “the lower aliphatic alcohol”, the above-described a liner or branched aliphatic alcohol having 1 to 5 carbon atoms can be used. Specific examples thereof include methanol, ethanol, 1-propanol, 2-propanol, 1-butanol, tert-butanol, 2-methyl-1-propanol, 2,2-dimethyl-1-propanol, 1,2-butanediol and 1,3-butanediol. Among them, 1-propanol, 1-butanol, 2,2-dimethyl-1-propanol, 1,2-butanediol and 1,3-butanediol are preferable. Any of these lower aliphatic alcohols may be mixed in the culture medium at an appropriate ratio.

As the carbon source, the lower aliphatic alcohols as mentioned above can be used. The amount of such a carbon source to be added to the culture medium is usually from about 0.1 to about 30% (w/v) of the culture solution.

Examples of the nitrogen source include natural organic nitrogen sources such as meat extract, peptone, yeast extract, malt extract, soybean flour, corn steep liquor, cottonseed flour, dry yeast and casamino acids; amino acids; sodium salts with inorganic acids such as sodium nitrate; ammonium salts with inorganic acids such as ammonium chloride, ammonium sulfate and ammonium phosphate; ammonium salts with organic acids such as ammonium fumarate and ammonium citrate; and urea. Among these nitrogen sources, the ammonium salts with organic acids, the natural organic nitrogen sources and amino acids may be used also as carbon sources in many cases. The amount of these nitrogen sources to be added to the culture medium is usually from about 0.1 to about 30% (w/v) of the culture solution.

Examples of the organic salt and the inorganic salt include chlorides, sulfates, acetates, carbonates and phosphates of potassium, sodium, magnesium, iron, manganese, cobalt and zinc. Specific examples thereof are sodium chloride, potassium chloride, magnesium sulfate, ferrous sulfate, manganese sulfate, cobalt chloride, zinc sulfate, copper sulfate, sodium acetate, calcium carbonate, potassium hydrogenphosphate and potassium dihydrogenphosphate. The amount of these organic salts and/or inorganic salts to be added to the culture medium is usually from about 0.0001 to about 5% (w/v) of the culture solution.

As the culture method, solid culture and liquid culture (e.g., test-tube culture, flask culture, or jar fermenter culture) are exemplified.

There is no particular limit in selection of a culture temperature and pH of a culture solution, insofar as these conditions enable culture of the present microorganism. For example, a culture temperature is from about 15° C. to about 45° C. and a pH of a culture solution is from about 4 to about 8. While a culture time may be optionally selected depending on culture conditions, it is usually from about 1 day to about 7 days.

It is possible to directly use the microbial cells of the present microorganism as a catalyst for use in the present oxidation reaction 2. As the methods for directly using the microbial cells of the present microorganism, (1) a method in which the culture solution is used directly, and (2) a method in which the microbial cells recovered by centrifugal separation of the culture solution or the wet microbial cells obtained after optionally washing the recovered cells with a buffer solution or water is used, are exemplified.

As the catalyst for use in the present oxidation reaction 2, a processed product of the present microorganism may be used. Examples of such a processed product include a product obtained by treating the microbial cells obtained by culture, with an organic solvent (e.g., acetone, ethanol); a product obtained by freeze-drying such cells; a product obtained by treating such cells with an alkali; a product obtained by physically or enzymatically grinding such cells; and crude enzyme separated and extracted from these products. The processed products further include products which are obtained by treating the cells as described above, and immobilizing the same by a known method.

Specifically, the cells of the present microorganism, and processed products thereof (e.g., cell-free extract, crude protein, purified protein and immobilized products thereof) can be used as the catalysts of the present oxidation reaction 2. Examples of the processed products of the microbial cells include a freeze-dried microorganism, a microorganism treated with an organic solvent, a dried microorganism, a ground microorganism, an autolysate of microorganism, an ultrasonically treated microorganism, a microorganism extract and an alkali-treated microorganism. As a method for obtaining immobilized microorganism, a carrier-binding method (i.e., a method of adsorbing the present enzyme or the like onto an inorganic carrier such as silica gel or ceramic, cellulose, or an ion-exchange resin), and an entrapment method (i.e., a method of entrapping the enzyme in a polymeric net structure of polyacrylamide, sulfur-containing polysaccharide gel (e.g., carageenan gel), alginic acid gel, agar gel or the like), are exemplified.

In view of industrial production with the use of the present microorganism, the use of the sterilized microorganism may be more advantageous than the use of untreated microorganism, in the point of the restriction in the production equipment. Examples of the method of sterilizing the microorganism include a physical sterilization method (e.g., heating, drying, freezing, light beams, ultrasonic waves, filtration or current-carrying), sterilization method with chemicals (e.g., alkali, acid, halogen, oxidant, sulfur, boron, arsenic, metal, alcohol, phenol, amine, sulfide, ether, aldehyde, ketone, cyan and antibiotic). In general, it is desirable to select such a treating method which does not deactivate “the ability of the present enzyme to preferentially oxidize a hydroxyl group of a sulfur-containing aminoalcohol compound” and which causes low residue and less pollution on the reaction system, from the above-described sterilization methods.

The present oxidation reaction 2 is usually carried out in the presence of water. In this case, water may be in the form of a buffer solution. Examples of a buffering agent for use in the buffer solution include salts of alkali metals with phosphoric acid such as sodium phosphate and potassium phosphate; and salts of alkali metals with acetic acid such as sodium acetate and potassium acetate. Examples of an alkaline buffer solution include Tris-hydrochloric buffer solution, Tris-citric buffer solution and the like.

Furthermore, the present oxidation reaction 2 may be carried out in the presence of water and a hydrophobic organic solvent. In this case, examples of the hydrophobic organic solvent include ester solvents such as ethyl formate, ethyl acetate, propyl acetate, butyl acetate, ethyl propionate and butyl propionate; alcohol solvents such as n-butyl alcohol, n-amyl alcohol and n-octyl alcohol; aromatic hydrocarbon solvents such as benzene, toluene and xylene; ether solvents such as diethyl ether, diisopropyl ether and methyl-t-butyl ether; halogenated hydrocarbon solvents such as chloroform and 1,2-dichloroethane; and mixtures thereof.

Alternatively, the present oxidation reaction 2 may be carried out in the presence of water and a hydrophilic organic solvent. In this case, examples of the hydrophilic organic solvent include alcohol solvents such as methanol and ethanol; ketone solvents such as acetone; ether solvents such as dimethoxyethane, tetrahydrofuran and dioxane; dimethyl sulfoxide; and mixtures thereof.

The present oxidation reaction 2 is usually carried out at a pH of 3 to 11 in the water phase, but the pH may be appropriately varied insofar as the reaction is permitted to proceed. The reaction is carried out preferably on the alkali side, more preferably at a pH of 8 to 10 in the water layer.

The present oxidation reaction 2 is usually carried out at a temperature of from about 0 to about 60° C., but the temperature may be optionally varied insofar as the reaction is permitted to proceed.

The present oxidation reaction 2 is usually carried out for about 0.5 hour to about 10 days. The termination of the reaction can be confirmed, for example, by determining the amount of 2-amino-4-methylthio-1-butanol in the reaction solution by liquid chromatography, gas chromatography or the like, after completion of the addition of 2-amino-4-methylthio-1-butanol as a raw material compound.

A concentration of 2-amino-4-methylthio-1-butanol as the raw material compound in the present oxidation product 2 is usually 50% (w/v) or less. To keep the concentration of 2-amino-4-methylthio-1-butanol in the reaction system substantially constant, 2-amino-4-methylthio-1-butanol may be continuously or sequentially added to the reaction system.

In the present oxidation reaction 2, for example, saccharides such as glucose, sucrose and fructose or a surfactant such as TritonX-100, Tween 60 optionally may be added to the reaction system.

Recovery of methionine from the reaction solution may be done by a known method.

For example, a post-treatment such as an operation of extracting the organic solvent from the reaction solution, an operation of concentrating the solution, ion-exchange, crystallization or the like may be optionally combined with column chromatography, distillation or the like to thereby purify methionine.

Methionine obtained by the production process of the present invention may be in the form of a salt.

### EXAMPLES

Hereinafter, the present invention will be described in more detail by way of Examples.

### Production of 2-amino-3-buten-1-ol

A 100 mL stainless steel-made reaction tube with a magnetic rotor was charged with 1,2-epoxy-3-butene (200 mg), a 28% by weight of ammonia water (10 g) and scandium triflate (14 mg) to prepare a mixture thereof. The mixture was stirred at an internal temperature of 30° C. for 6 hours to react 1,2-epoxy-3-butene with ammonia. An internal pressure of the reaction tube was maintained at 0.3 to 0.4 MPa during the reaction. After completion of the reaction, the reaction mixture was cooled to a room temperature, and then, ammonia was evaporated from the reaction mixture. A part of the mixture obtained after the evaporation of ammonia was collected and was then analyzed by the gas chromatography internal standard method to determine the content of 2-amino-3-buten-1-ol, 1-amino-3-butene-2-ol and 1,2-epoxy-3-butene, and the yields thereof were calculated. In this regard, the contents of 2-amino-3-buten-1-ol and 1-amino-3-butene-2-ol were determined in terms of diacyl forms thereof by way of conversion thereof into diacyl forms by the use of acetyl chloride and pyridine:

Yield of 2-amino-3-buten-1-ol: 55%

Yield of 1-amino-3-butene-2-ol: 43%

Recovery rate of 1,2-epoxy-3-butene (as a raw material): 0%

### First Step

### Example 1-1

A 50 mL pressure reaction tube with a magnetic rotor was charged with 2-amino-3-buten-1-ol (100 mg), ethyl acetate (2 g) and 2,2′-azobis(4-methoxy-2,4-dimethylvaleronitrile) (10 mg) to prepare a mixture thereof. The mixture was cooled to an internal temperature of −20° C., and then, methanethiol (500 mg) was added to the mixture. After the reaction tube was tightly sealed, the temperature was elevated to 40° C., and then, the mixture was stirred at 40° C. for 4 hours. An internal pressure (i.e., a gauge pressure) of the reaction tube determined just after the temperature elevation to 40° C. was 2 kg/cm² (equivalent to 0.20 MPa); and the same pressure determined after the stirring of the mixture at 40° C. for 4 hours was 1 kg/cm² (equivalent to 0.10 MPa). After completion of the reaction, non-reacted methanethiol was removed by blowing a nitrogen gas into the resultant reaction mixture. A part of the mixture obtained after the removal of methanethiol was collected and was then analyzed by the gas chromatography internal standard method to determine the content of 2-amino-4-methylthio-1-butanol, and the yield thereof was calculated. The yield of 2-amino-4-methylthio-1-butanol was 90%. 5% of 2-amino-3-buten-1-ol used as the raw material was recovered.

### Example 1-2

A 50 mL pressure reaction tube with a magnetic rotor was charged with 2-amino-3-buten-1-ol (300 mg), ethyl acetate (3 g) and 2,2′-azobis(4-methoxy-2,4-dimethylvaleronitrile) (10 mg) to prepare a mixture thereof. The mixture was cooled to an internal temperature of −20° C., and then, methanethiol (1.0 g) was added to the mixture. After the reaction tube was tightly sealed, the temperature was elevated to 40° C., and then, the mixture was stirred at 40° C. for 4 hours. An internal pressure (i.e., a gauge pressure) of the reaction tube determined just after the temperature elevation to 40° C. was 3 kg/cm² (equivalent to 0.30 MPa); and the same pressure determined after the stirring of the mixture at 40° C. for 4 hours was 1 kg/cm² (equivalent to 0.10 MPa). After completion of the reaction, non-reacted methanethiol was removed by blowing a nitrogen gas into the resultant reaction mixture. A part of the mixture obtained after the removal of methanethiol was collected and was then analyzed by the gas chromatography internal standard method to determine the content of 2-amino-4-methylthio-1-butanol, and the yield thereof was calculated. The yield of 2-amino-4-methylthio-1-butanol was 91%. 5% of 2-amino-3-buten-1-ol used as the raw material was recovered.

450 mg of colorless liquid of 2-amino-4-methylthio-1-butanol was obtained by concentrating the mixture obtained after the removal of methanethiol. The colorless liquid was solidified in a freezer (−10° C.)

### Second Step

### Example 2-1

A 50 mL pressure reaction tube with a magnetic rotor was charged with 2-amino-4-methylthio-1-butanol (200 mg), sodium hydroxide (90 mg) and water (2 g) to prepare a mixture thereof. A sponge copper (Raney (registered trademark) type, manufactured by Strem Chemical Inc.) (40 mg) was added to the mixture. The interior of the reaction tube was purged by a nitrogen gas, and then, the mixture was heated to 140° C. and was then stirred at 140° C. for 8 hours. After the reaction mixture was cooled to a room temperature, the sponge copper was removed from the reaction mixture by filtering the reaction mixture. The resulting filtrate was neutralized by adding 0.1N sulfuric acid thereto, and then, water was distilled off. Thus, 2-amino-4-(methylthio)butyric acid, i.e., methionine was obtained.

### Determination of Yield:

Methanol (5 g) was added to the obtained 2-amino-4-(methylthio)butyric acid, and a 10% by weight of hexane solution of trimethylsilyldiazomethane was further added thereto, to obtain methyl 2-amino-4-(methylthio)butyrate. A part of the resulting methanol solution containing methyl 2-amino-4-(methylthio)butyrate was collected and was then analyzed by a gas chromatography internal standard method to determine the yield of methyl 2-amino-4-(methylthio)butyrate from 2-amino-4-methylthio-1-butanol. As a result, the yield was 37%. In other words, 2-amino-4-(methylthio)butyric acid was obtained at a yield of 37% or more from 2-amino-4-methylthio-1-butanol. 49% of 2-amino-4-methylthio-1-butanol used as the raw material was recovered.

### Example 2-2

A 50 mL pressure reaction tube with a magnetic rotor was charged with 2-amino-4-methylthio-1-butanol (200 mg), sodium hydroxide (120 mg) and water (2 g), and the mixture was stirred. A sponge copper (Raney (registered trademark) type, manufactured by Strem Chemical Inc.) (50 mg) was added to the mixture as a developing catalyst. The interior of the reaction tube was purged by a nitrogen gas, and then, the mixture was heated to 140° C. and was then stirred at 140° C. for 8 hours. After the reaction mixture was cooled to a room temperature, the sponge copper was removed from the reaction mixture by filtering the reaction mixture. Ethyl acetate (5 g) was added to the resulting filtrate to separate oil and water, and thus the lipophilic substances were removed therefrom. Carbonic acid was formed by adding dry ice (CO₂) (5 g) to the water phase, and a solid was precipitated upon stirring. The precipitated solid was filtered and dried to obtain a white powder (130 mg). Then, the obtained powder was analyzed by a liquid chromatography (modified area percentage method). As a result, the content of 2-amino-4-(methylthio)butyric acid was 64%. The yield of 2-amino-4-(methylthio)butyric acid from 2-amino-4-methylthio-1-butanol was 38%.

### Example 2-3

A 50 mL pressure reaction tube with a magnetic rotor was charged with 2-amino-4-methylthio-1-butanol (135 mg), sodium hydroxide (40 mg), water (1 g), acetonitrile (1 g) and a 5% by weight of Pt/C (containing 50% by weight of water) (100 mg), and the reaction tube was pressurized to 1 MPa with air. The mixture was heated to 50° C. and was then stirred at 50° C. for 8 hours. After the reaction mixture was cooled to a room temperature, the Pt/C was removed from the reaction mixture by filtering the reaction mixture. The resulting filtrate was neutralized by adding 0.1N sulfuric acid thereto, and then, the solvent was distilled off. Thus, 2-amino-4-(methylthio) butyric acid, i.e., methionine was obtained.

### Determination of Yield:

Methanol (5 g) was added to the obtained 2-amino-4-(methylthio)butyric acid, and a 10% by weight of hexane solution of trimethylsilyldiazomethane was further added thereto, to obtain methyl 2-amino-4-(methylthio)butyrate. The resulting methanol solution containing methyl 2-amino-4-(methylthio)butyrate was analyzed by a gas chromatography internal standard method to determine the yield of methyl 2-amino-4-(methylthio)butyrate from 2-amino-4-methylthio-1-butanol. As a result, the yield was 14%. In other words, 2-amino-4-(methylthio)butyric acid was obtained at a yield of 14% or more from 2-amino-4-methylthio-1-butanol. 80% of 2-amino-4-methylthio-1-butanol used as the raw material was recovered.

### Example 2-4

A 50 mL pressure reaction tube with a magnetic rotor was charged with 2-amino-4-methylthio-1-butanol (100 mg), sodium bicarbonate (70 mg), acetonitrile (3 g) and a 5% by weight of Pt/C (containing 50% by weight of water) (100 mg), and the resulting mixture was stirred at 60° C. for 8 hours under an atmosphere of air. The reaction mixture was cooled to room temperature and was then filtered. The resulting filtrate was neutralized by adding 0.1N sulfuric acid thereto, and then, the solvent was distilled off. Thus, 2-amino-4-(methylthio) butyric acid was obtained.

### Determination of Yield:

Methanol (5 g) was added to the obtained 2-amino-4-(methylthio)butyric acid, and a 10% by weight of hexane solution of trimethylsilyldiazomethane was further added thereto, to obtain methyl 2-amino-4-(methylthio)butyrate. The resulting methanol solution containing methyl 2-amino-4-(methylthio)butyrate was analyzed by a gas chromatography internal standard method to determine the yield of methyl 2-amino-4-(methylthio)butyrate from 2-amino-4-methylthio-1-butanol. As a result, the yield was 9%. In other words, 2-amino-4-(methylthio)butyric acid was obtained at a yield of 9% or more from 2-amino-4-methylthio-1-butanol. 90% of 2-amino-4-methylthio-1-butanol used as the raw material was recovered.

### Example 2-5

A 50 mL pressure reaction tube with a magnetic rotor was charged with 2-amino-4-methylthio-1-butanol (100 mg), sodium bicarbonate (30 mg), water (1 g), acetonitrile (1 g) and a 5% by weight of Ru/C (containing 50% by weight of water) (50 mg), and the resulting mixture was stirred at 50° C. for 8 hours under an atmosphere of air. The reaction mixture was cooled to room temperature and then filtered. The resulting filtrate was neutralized by adding 0.1N sulfuric acid thereto, and then, the solvent was distilled off. Thus, 2-amino-4-(methylthio)butyric acid was obtained.

### Determination of Yield:

Methanol (5 g) was added to the obtained 2-amino-4-(methylthio)butyric acid, and a 10% by weight of hexane solution of trimethylsilyldiazomethane was further added thereto, to obtain methyl 2-amino-4-(methylthio)butyrate. The resulting methanol solution containing methyl 2-amino-4-(methylthio)butyrate was analyzed by a gas chromatography internal standard method to determine the yield of methyl 2-amino-4-(methylthio)butyrate from 2-amino-4-methylthio-1-butanol. As a result, the yield was 5%. In other words, 2-amino-4-(methylthio)butyric acid was obtained at a yield of 5% or more from 2-amino-4-methylthio-1-butanol. 90% of 2-amino-4-methylthio-1-butanol used as the raw material was recovered.

### Example 2-6

A 50 mL pressure reaction tube with a magnetic rotor was charged with 2-amino-4-methylthio-1-butanol (135 mg) obtained by the Example 1-2, sodium hydroxide (80 mg), water (1 g), acetonitrile (1 g) and a 5% by weight of Pt/C (containing 50% by weight of water) (100 mg), and the reaction tube was pressurized to 1 MPa with air. The resulting mixture was heated to 50° C. and was then stirred at 50° C. for 8 hours. After the reaction mixture was cooled to a room temperature, the Pt/C was removed from the reaction mixture by filtering the reaction mixture. The resulting filtrate was neutralized by adding 0.1N sulfuric acid thereto, and then, the solvent was distilled off. Thus, 2-amino-4-(methylthio)butyric acid, i.e., methionine was obtained.

### Determination of Yield:

Methanol (5 g) was added to the obtained 2-amino-4-(methylthio)butyric acid, and a 10% by weight of hexane solution of trimethylsilyldiazomethane was further added thereto, to obtain methyl 2-amino-4-(methylthio)butyrate. The resulting methanol solution containing methyl 2-amino-4-(methylthio)butyrate was analyzed by a gas chromatography internal standard method to determine the yield of methyl 2-amino-4-(methylthio)butyrate from 2-amino-4-methylthio-1-butanol. As a result, the yield was 6%. In other words, 2-amino-4-(methylthio)butyric acid was obtained at a yield of 6% or more from 2-amino-4-methylthio-1-butanol. 78% of 2-amino-4-methylthio-1-butanol used as the raw material was recovered.

### Search of Microorganism with Ability to Convert 2-Amino-4-Methylthio-1-Butanol into Methionine

### Reference Example 1

A test tube is charged with a sterilized culture medium (which has been prepared by adding, to water, polypeptone, yeast extract, meat extract, ammonium sulfate, potassium dihydrogenphosphate and magnesium sulfate heptahydrate, and adjusting the pH of the resulting mixture to 7.0). Then, the microbial cells obtained from the culture collections or the microbial cells prepared by purely separation from soil are inoculated on this culture medium. The cells are subjected to shaking culture at 30° C. under an aerobic condition. After completion of the culture, the cells are recovered as viable cells by centrifugal separation. A screw-top test tube is charged with 0.1M Tris-glycine buffer (pH 10), and the viable cells are added, and then they are suspended in each other. The resulting suspension is admixed with 2-amino-4-methylthio-1-butanol, and the resulting mixture is shaken at 30° C. for 3 to 7 days.

After completion of the reaction, the reaction solution is sampled. The microbial cells are removed from this sampling solution, and then, an amount of produced methionine is analyzed by liquid chromatography.

In this way, a microorganism capable of converting 2-amino-4-methylthio-1-butanol into methionine is screened.

### Condition for Content-Analyzing:

Column: Cadenza CD-C18 (4.6 mmφ×15 cm, 3 μm)
(manufactured by Imtakt Corporation)

Mobile Phase:
Solution A: an aqueous solution of 0.1% trifluoroacetic acid
Solution B: methanol

Time (min.) Solution A (%): Solution B (%)

| | |
|---|---|
| 0 | 100:0 |
| 10 | 100:0 |
| 20 | 50:50 |
| 25 | 50:50 |
| 25.1 | 100:20 |

Flow rate: 0.5 ml/min.

Column temperature: 40° C.

Detection: 220 nm

### Examples 2-7 to 2-26

A culture medium was prepared by adding, to water (1 L), a lower aliphatic alcohol (5 g) listed in the following Table 1 to 4, polypeptone (5 g), yeast extract (3 g), meat extract 3 g), ammonium sulfate (0.2 g), potassium dihydrogenphosphate (1 g) and magnesium sulfate heptahydrate (0.5 g), adjusting the pH of the resulting mixture to 7.0, and sterilizing the resulting mixture. A test tube was charged with the sterilized culture medium (5 g), and then, the cells of Rhodococcus rhodochrous ATCC19149 (Examples 2-7 to 2-11 of Table 1), Rhodococcus rhodochrous ATCC19150 (Examples 2-12 to 2-16 of Table 2), Rhodococcus sp. ATCC19070 (Examples 2-17 to 2-21 of Table 3) or Rhodococcus sp. ATCC19148 (Examples 2-22 to 2-26 of Table 4) were inoculated on this culture medium. The cells were subjected to shaking culture at 30° C. under an aerobic condition. After completion of the culture, the cells were recovered as viable cells by centrifugal separation. A screw-top test tube was charged with 0.1M Tris-glycine buffer (pH 10), and the viable cells were added, and then, they were suspended in the buffer. The resulting suspension was admixed with 2-amino-4-methylthio-1-butanol (2 mg) obtained by the Example 1-2, and the resulting mixture was shaken at 30° C. for 7 days.

After completion of the reaction, 0.5 ml of the reaction solution was sampled. The microbial cells were removed from this sampling solution, and then, an amount of produced methionine was analyzed by liquid chromatography.

The results are shown in Tables 1 to 4.

### Condition for Content-Analyzing:

Column: Cadenza CD-C18 (4.6 mmφ×15 cm, 3 μm)
(manufactured by Imtakt Corporation)

Mobile Phase:
Solution A: an aqueous solution of 0.1% trifluoroacetic acid
Solution B: methanol

Time (min.) Solution A (%): Solution B (%)

| | |
|---|---|
| 0 | 100:0 |
| 10 | 100:0 |
| 20 | 50:50 |
| 25 | 50:50 |
| 25.1 | 100:0 |

Flow rate: 0.5 ml/min.

Column temperature: 40° C.

Detection: 220 nm

TABLE 1
Rhodococcus rhodochrous ATCC19149

| | Lower aliphatic alcohol added | |
|---|---|---|
| Examples | in the culture medium | Yield of Methionine (%) |
| 2-7 | 1-propanol | 24.2 |
| 2-8 | 1-butanol | 56.6 |
| 2-9 | 1,2-butanediol | 63.8 |
| 2-10 | 2,2-dimethyl-1-propanol | 17.5 |
| 2-11 | 1,3-butanediol | 81.6 |

TABLE 2
Rhodococcus rhodochrous ATCC19150

| | Lower aliphatic alcohol added | |
|---|---|---|
| Examples | in the culture medium | Yield of Methionine (%) |
| 2-12 | 1-propanol | 33.6 |
| 2-13 | 1-butanol | 54.8 |
| 2-14 | 1,2-butanediol | 67.5 |
| 2-15 | 2,2-dimethyl-1-propanol | 39.9 |
| 2-16 | 1,3-butanediol | 69.8 |

TABLE 3
Rhodococcus sp. ATCC19070

| | Lower aliphatic alcohol added | |
|---|---|---|
| Examples | in the culture medium | Yield of Methionine (%) |
| 2-17 | 1-propanol | 60.7 |
| 2-18 | 1-butanol | 96.4 |
| 2-19 | 1,2-butanediol | 60.0 |
| 2-20 | 2,2-dimethyl-1-propanol | 27.1 |
| 2-21 | 1,3-butanediol | 60.6 |

TABLE 4
Rhodococcus sp. ATCC19148

| | Lower aliphatic alcohol added | |
|---|---|---|
| Examples | in the culture medium | Yield of Methionine (%) |
| 2-22 | 1-propanol | 28.7 |
| 2-23 | 1-butanol | 37.7 |
| 2-24 | 1,2-butanediol | 27.9 |
| 2-25 | 2,2-dimethyl-1-propanol | 30.6 |
| 2-26 | 1,3-butanediol | 59.3 |

### INDUSTRIAL APPLICABILITY

The present invention can be utilized as a process for producing methionine which is an essential amino acid and is a very useful for a feed additive.

1. A process for producing methionine, comprising a first step of reacting 2-amino-3-buten-1-ol with methanethiol, and a second step of oxidizing 2-amino-4-methylthio-1-butanol obtained in the first step.

2. The process according to claim 1, wherein the first step is a step of reacting 2-amino-3-buten-1-ol with methanethiol in the presence of a radical initiator.

3. The process according to claim 2, wherein the radical initiator is an azo compound.

4. The process according to claim 1, wherein the first step is a step of reacting 2-amino-3-buten-1-ol with methanethiol in the presence of a solvent.

5. The process according to claim 4, wherein the solvent is an ester solvent.

6. The process according to claim 1, wherein the second step is a step of oxidizing 2-amino-4-methylthio-1-butanol in the presence of at least one metal selected from the group consisting of copper and the elements belonging to Group 8, 9 or 10 of the periodic table.

7. The process according to claim 1, wherein the second step is a step of oxidizing 2-amino-4-methylthio-1-butanol in the presence of copper and water.

8. The process according to claim 1, wherein the second step is a step of oxidizing 2-amino-4-methylthio-1-butanol in the presence of oxygen and either ruthenium or platinum.

9. The process according to claim 6, wherein the second step is a step of oxidizing 2-amino-4-methylthio-1-butanol further in the presence of at least one typical metal compound selected from the group consisting of alkali metal compounds and alkaline earth metal compounds.

10. The process according to the claim 9, wherein the typical metal compound is an alkali metal hydroxide or an alkaline earth metal hydroxide.

11. The process according to claim 1, wherein the second step is a step of oxidizing 2-amino-4-methylthio-1-butanol by an action of a microbial cell of a microorganism capable of converting 2-amino-4-methylthio-1-butanol into methionine or by an action of a processed product of the microbial cell.

12. The process according to claim 11, wherein the microorganism is a microorganism cultured in a culture medium containing a lower aliphatic alcohol.

13. The process according to claim 12, wherein the lower aliphatic alcohol is a liner or branched aliphatic alcohol having 1 to 5 carbon atoms.

14. The process according to claim 11, wherein the microorganism is at least one microorganism selected from the group consisting of the microorganisms belonging to the genus Alcaligenes, the microorganisms belonging to the genus Bacillus, the microorganisms belonging to the genus Pseudomonas, the microorganisms belonging to the genus Rhodobacter and the microorganisms belonging to the genus Rhodococcus.

15. A process for producing 2-amino-4-methylthio-1-butanol, comprising a step of reacting 2-amino-3-buten-1-ol with methanethiol.

16. The process according to claim 15, wherein the above-described step is a step of reacting 2-amino-3-buten-1-ol with methanethiol in the presence of a radical initiator.

17. The process according to claim 16, wherein the radical initiator is an azo compound.

18. The process according to claim 15, wherein said step is a step of reacting 2-amino-3-buten-1-ol with methanethiol in the presence of a solvent.

19. The process according to claim 18, wherein the solvent is an ester solvent.
